(19) United States
(12) Patent Application Publication
(10) Pub. No.: US 2023/0395207 A1
(43) Pub. Date: Dec. 7, 2023

(54) A METHOD USED FOR DETERMINING PERSONALIZED REFERENCE RANGE OF MEDICAL LABORATORY TESTS

(71) Applicant: ACIBADEM MEHMET ALI AYDINLAR UNIVERSITESI, Istanbul (TR)

(72) Inventors: Abdurrahman COSKUN, Istanbul (TR); Sverre SANDBERG, Bergen (NO); Ibrahim UNSAL, Istanbul (TR); Coskun CAVUSOGLU, Istanbul (TR); Mustafa SERTESER, Istanbul (TR); Meltem KILERCIK, Istanbul (TR); Aasne Karine AARSAND, Bergen (NO)

(73) Assignee: ACIBADEM MEHMET ALI AYDINLAR UNIVERSITESI, Istanbul (TR)

(21) Appl. No.: 18/031,764

(22) PCT Filed: Nov. 13, 2020
(86) PCT No.: PCT/TR20/51103

§ 371 Date: Apr. 13, 2023

(30) Foreign Application Priority Data
Oct. 14, 2020 (TR) 2020/16430

(51) Int. Cl. G16H 10/40; G16H 50/30

(52) U.S. Cl. CPC G16H 10/40; G16H 50/30

(57) ABSTRACT

The present invention relates to a method for calculating a personal reference range in order to compare results of a measurement carried out in case of suspicion of an illness, by using results of persons'laboratory tests measured in a period when they feel healthy.

### TECHNICAL FIELD

The present invention relates to a method for calculating a personal reference range in order to compare results of a measurement carried out in case of suspicion of an illness, by using results of persons' laboratory tests measured in a period when they feel healthy.

### BACKGROUND OF THE INVENTION

Persons who have their laboratory tests measured compare their results with a range upper and lower limits of which are determined according to certain statistical rules, and which does not belong to them and is usually called as population-based reference ranges. However, due to the fact that population-based reference ranges do not entirely reflect the individual's condition and physiological change, they may lead to occurrence of medical errors in interpreting patient results. Even though the laboratory results of a person are within population-based reference ranges, the person may not be healthy or the person can be healthy even though his/her laboratory results are out of population-based reference ranges. Therefore, although reference ranges used today and based on population are obtained from persons with similar characteristics, they do not entirely represent individuals and lead to occurrence of medical errors. For this reason, there is need for solutions whereby medical errors arising from reference ranges are avoided and different reference ranges are created for each individual in a personalized way by using persons' own self-data for interpretation of laboratory results of persons today.

The United States patent document no. US20160282361 discloses a method for carrying out mass spectrometric analysis of peptides. The method in the said invention enables interpretation of mass spectrometric tests for clinical biomarker peptides and addition of stable isotope labeled peptides to peptide proportioning. Only personal reference values of patients whose peptide mass spectrometric analysis tests are conducted and their personal reference ranges are mentioned in the said invention only discloses

The Japanese patent document no. JP2018536846A, another application in the state of the art, discloses a method for personalizing biomarker signals and using personalized biomarker signals in medical procedures. In addition to intra-individual and inter-individual biological variation values of personalized biomarkers and analytical mean and standard deviation values of tests conducted in a period when the patient was healthy, a variation value is used for personalizing biomarker signals and for determining personalized biomarker value range by using Bayesian method in the said invention.

### SUMMARY OF THE INVENTION

An objective of the present invention is to realize a method which uses arithmetic mean of measurement results of a laboratory test of a user that is measured in a certain range and number, preferably on different days (at least three measurements) in a period when s/he is healthy and the variation values (analytical and biological) of the related test, and enables to determine personal reference range of persons/users for the related test on the basis of the said values.

### DETAILED DESCRIPTION OF THE INVENTION

“A Method Used for Determining Personalized Reference Range of Medical Laboratory Tests” realized to fulfil the objective of the present invention is shown in the FIGURE attached, in which:

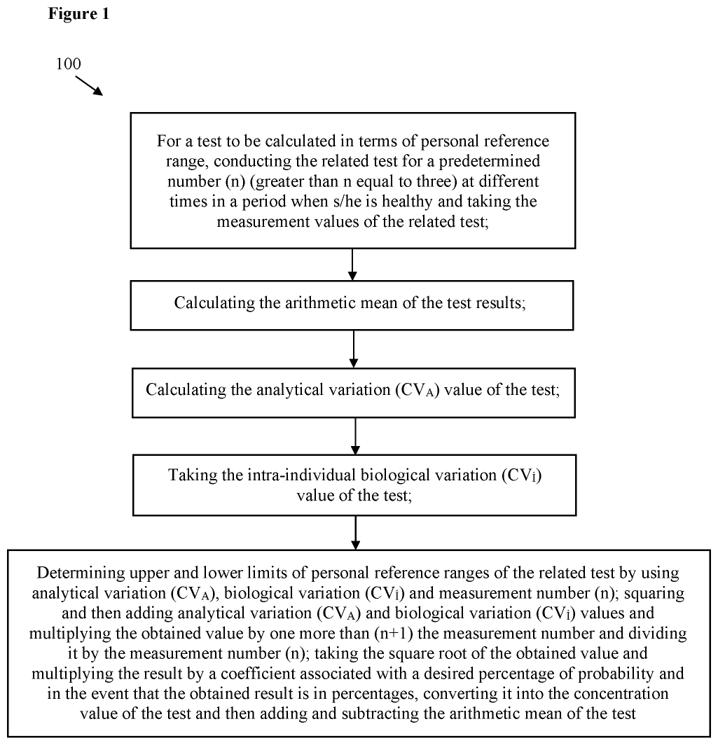

FIG. 1 is a view of flowchart steps of the inventive method used for determining personalized reference range of medical laboratory tests.

100. Method

The symbols used in the detailed description part refer to the following:

TV (total variation): Total variation around homoestatic set point

Z: Probability Coefficient

n: Measurement number of the test

CV: Coefficient of variation

CV_{A}: Analytical variation of the test

CV_{İ} Intra-individual biological variation of the test

köRA: Personal reference range

X̅: Arithmetic mean of measurement results

The inventive method (100) used for calculating personal reference ranges (köRA) of medical laboratory tests comprises steps of:
for a test to be calculated in terms of personal reference range, conducting the related test for a predetermined number (n) (greater than n equal to three) at different times in a period when s/he is healthy and taking the measurement values of the related test;
calculating the arithmetic mean of the test results;
calculating the analytical variation (CV_{A}) value of the test;
taking the intra-individual biological variation (CV_{İ}) value of the test;
determining upper and lower limits of personal reference ranges of the related test by using analytical variation (CV_{A}), biological variation (CV_{İ}) and number of measurement (n); squaring and then adding analytical variation (CV_{A}) and biological variation (CV_{İ}) values and multiplying the obtained value by one more than (n+1) the number of measurement and dividing it by the number of measurement (n); taking the square root of the obtained value and multiplying the result by a coefficient associated with a desired percentage of probability and in the event that the obtained result is in percentages, converting it into the concentration value of the test and then adding and subtracting it to/from the arithmetic mean of the test.

In the inventive method (100), tests—the reference range of which is aimed to be determined—are chosen, it is accepted that the person does not have any disease symptom that will affect the test result and the person does not take any medicine or medicines to affect the test result during measurement. Sample is taken from person/persons from whom it is aimed to determine the reference range, on different days and preferably at the same time of the day for n times (n≥3) and the measurements obtained in the test results are taken.

In the inventive method (100), measurement of the related test or tests are carried out in the samples taken in different periods of time for n (n≥3) times and results of the measurement carried out are recorded. The measurement results taken are used for determining homoestatic set point of the related test or tests.

In the inventive method (100), sum of the measurement results are divided by the number of measurements by using the test results in order to calculate personal reference ranges at first. Then the analytical variation (CV_{A}) of the test is calculated and its biological variation (CV_{İ}) value is received from a biological variation database. In another preferred embodiment, cumulative analytical variation value can be used instead of CV_{A} in the method (100). In a further preferred embodiment, CV_{İ} value can be used as standard deviation (SD) value.

In the method (100), after analytical variation (CV_{A}) and biological variation (CV_{İ}) values are obtained, total variation around the homeostatic set point is determined by using the said values and the number of measurements (n). In a preferred embodiment, total variation is determined for 95% probability. Thus, a certain value is calculated by means of a calculation wherein analytical variation (CV_{A}), biological variation (CV_{İ}) and number of measurements (n) of the test are included around the arithmetic mean calculated. In the inventive method (100), in order to calculate the total variation (TV) around the homoestatic set point: analytical and biological variations (CV_{A}, CV_{İ}) of the test are squared and then added; these values added are multiplied by one more than (n+1) the number of measurement and divided by the number of measurement (n); then the square root of the obtained value is taken; and lastly the obtained result is multiplied by a z value at first. The z value included in the said calculation is preferably taken as 1.96 for 95% probability, whereas different z values can be used for different probabilities. (I)

$$T V = z \times \sqrt{\frac{( n + 1 )}{n} \times ( {CV}_{A}^{2} + C {V}_{I}^{2} )}$$

The personal reference range of the related test calculated by the inventive method (100) indicates the area up to the total variation value around the arithmetic mean calculated by 95% probability. Thereby, arithmetic mean±total variation area provides the personal reference range of the related test.

In order that the values obtained by the inventive method (100) can be converted from percentage into concentration value (köRA), the concentration limit values of the area (köRA) are obtained after multiplying the value of arithmetic mean of measurement results (X̅) by the value of total variation around homoestatic set point and dividing by 100 and then adding and subtracting it to/from the value of arithmetic mean of measurement results (X̅).

köRA=X̅±(TV×X̅)/100 (II)

In the inventive method (100), the standard variation of homoestatic set point indicate the central change of personal reference range. Distribution of test results around homeostatic set point refers to the area wherein the test results can be included within a predetermined probability around the homoestatic set point of the test results. The fact that the said test results can be included within a predetermined probability around the homoestatic set point of the test results is obtained by combining the intra-individual biological variation (CV_{İ}) and the analytical variation (CV_{A}) values of the test through Gaussian method by variation of the homeostatic set point of the area limits and multiplying the obtained value by a z factor. The value calculated by the inventive method (100) (for 95% probability) is also accepted as the total variation. The personal reference range (köRA) calculated by the inventive method (100) indicates the area whereby the result of the related test that the person had when s/he was healthy could be obtained by a determined percentage of probability.

Potential error sources that may occur for patients are avoided by personal reference ranges (köRA) which can also indicate the values—that are calculated by taking account of population values and not healthy for the person—as normal and which are calculated by means of the inventive method (100) instead of broader and population-based reference ranges. Reference values detected within a specific probability of an individual are obtained by means of the personal reference ranges (köRA).

Within these basic concepts; it is possible to develop various embodiments of the inventive method used for calculating personal reference range (1); the invention cannot be limited to examples disclosed herein and it is essentially according to claims.

1. A method used for calculating personal reference ranges (koRA) of medical laboratory tests; characterized by comprising steps of:
for a test to be calculated in terms of personal reference range, conducting the related test for a predetermined number (n) (greater than n equal to three) at different times in a period when s/he is healthy and taking the measurement values of the related test;
calculating the arithmetic mean of the test results;
calculating the analytical variation (CVA) value of the test;
taking the intra-individual biological variation (CVt) value of the test;
determining upper and lower limits of personal reference ranges of the related test by using analytical variation (CVA), biological variation (CVt) and number of measurement (n); squaring and then adding analytical variation (CVA) and biological variation (CVt) values and multiplying the obtained value by one more than (n+1) the number of measurement and dividing it by the number of measurement (n); taking the square root of the obtained value and multiplying the result by a coefficient associated with a desired percentage of probability and in the event that the obtained result is in percentages, converting it into the concentration value of the test and then adding and subtracting it to/from the arithmetic mean of the test.

2. The method according to claim 1; characterized in that sample is taken from person/persons from whom it is aimed to determine the reference range, on different days and preferably at the same time of the day for n (n>three) times and the measurements obtained in the test results are taken.

3. The method according to claim 1; characterized in that sum of the measurement results are divided by the number of measurements by using the test results in order to calculate personal reference ranges at first.

4. The method according to claim 1; characterized in that after analytical variation (CVA) and biological variation (CVt) values are obtained, total variation around the homeostatic set point is determined by using the said values and the number of measurements (n).

5. The method according to claim 1; characterized in that in order to calculate the total variation (TV) around the homoestatic set point: analytical and biological variations (CVA, CVt) of the test are squared and then added; these values added are multiplied by one more than (n+1) the number of measurement and divided by the number of measurement (n); then the square root of the obtained value is taken; and lastly the obtained result is multiplied by a z value at first.

6. The method according to claim 1; characterized in that in order that the values can be converted from percentage into concentration value (kbRA), the concentration limit values of the area (kbRA) are obtained after multiplying the value of arithmetic mean of measurement results (X) by the value of total variation around homoestatic set point and dividing by 100 and then adding and subtracting it to/from the value of arithmetic mean of measurement results (X).
